(19) United States
(12) Patent Application Publication
(10) Pub. No.: US 2022/0160291 A1
(43) Pub. Date: May 26, 2022

(54) SYSTEM FOR RECORDING OF SEIZURES

(71) Applicant: MOCXA Health Private Limited, Bangalore (IN)

(72) Inventors: Raja Aditya Kadambi, Mahadevapura (IN); Ankita Kumar, Mahadevapura (IN)

(21) Appl. No.: 17/303,562

(22) Filed: Jun. 2, 2021

(30) Foreign Application Priority Data
Nov. 23, 2020 (IN) 202041050987

(51) Int. Cl. A61B 5/00; A61B 5/369; A61B 5/11; A61B 5/291

(52) U.S. Cl. CPC A61B 5/4094; A61B 5/369; A61B 5/1128; A61B 2562/0219; A61B 5/0006; A61B 5/7267; A61B 5/291

(57) ABSTRACT

A system includes an EEG headset, comprising EEG electrodes and InfraRed (IR) transmitters, to be worn by a subject. A recording unit is communicably coupled to the EEG headset and comprises a computing device for receiving EEG feed from the EEG headset, IR coordinates corresponding to the IR transmitters from an IR sensor, and a video recording of the subject from a video camera. The computing device is to identify a position of the subject based on the IR coordinates or the video recording or a combination thereof; cause the computing device to be reoriented based on the position of the subject; and facilitate detection and recording of seizure events based on the EEG feed or video recording or IR coordinates or a combination thereof. The computing device provides a seizure event report based on detection of the seizure events.

### CLAIM OF PRIORITY

This application claims the benefit of priority to Indian Application No. 202041050987, filed 23 Nov. 2020, which application is incorporated by reference as if reproduced herein and made a part hereof in its entirety, and the benefit of priority of which is claimed herein.

### TECHNICAL FIELD

The present subject matter relates, in general, to recording of seizures and, in particular, to a system for recording of seizures with a video Electroencephalogram (EEG).

### BACKGROUND

Seizures are neurological disorders caused by electrical disturbances occurring in the brain of an individual. Seizures occurring recurrently, called epilepsy, can be severely debilitating and/or dangerous. Epilepsy is characterized by the occurrence of seizures, episodic impairment, loss of consciousness, abnormal motor phenomena, and psychic or sensory disturbances. An electroencephalogram (EEG) test is used for detecting abnormalities related to electrical activities of the brain. An EEG tracks and records brain wave patterns. In this procedure, conventionally, small metal discs with thin wires (electrodes) are placed on the scalp of the individual and wires from the electrodes are connected to the electroencephalograph (EEG) machine, which then sends signals to a computer to process and record the results. The EEG machine detects and amplifies the electrical signals and records them onto a paper or a computer. The electrodes are removed at the end of the test. This procedure is typically conducted in a healthcare facility or a diagnostic laboratory.

Video EEG test records the electrical activity of the brain on an EEG and simultaneously records a video of the physical manifestation of the seizure activity of the individual. A neurologist can read the EEG chart and view the video of the seizure to determine the nature of the seizure and diagnose it. Multiple sessions may often be required to reach the best diagnosis. Continuous monitoring may be more effective in cases where there are frequent seizures, or when the seizure activity is triggered in a diagnostic setting, for example, by using flashing lights. However, generally, an attendant has to be present to ensure the recording is started when a seizure event occurs and to change the position of the camera in case the subject moves out of its field of view, leading to increased costs and logistical complexities.

### BRIEF DESCRIPTION OF FIGURES

The detailed description is provided with reference to the accompanying figures. In the figures, the left-most digit(s) of a reference number identifies the figure in which the reference number first appears. The same numbers are used throughout the figures to reference like features and components.

FIG. 1 illustrates a conventional system for recording of seizure.

FIG. 2 illustrates a block diagram of a system for recording of seizure, in accordance with an example implementation of the present subject matter.

FIG. 3a illustrates recording of seizure using a recording unit, in accordance with an example implementation of the present subject matter.

FIG. 3b illustrates a tabletop recording unit, in accordance with an example implementation of the present subject matter

FIGS. 4a and 4b depict a flowchart for recording of seizure, in accordance with an example implementation of the present subject matter.

FIG. 5 is a schematic representation of a tracker of an EEG headset, in accordance with an example implementation of the present subject matter.

FIGS. 6a-6d depict pictorial views of tracking horizontal motion of a subject, in accordance with an example implementation of the present subject matter.

FIGS. 7a-7d depict pictorial views of tracking vertical motion of a subject, in accordance with an example implementation of the present subject matter.

FIG. 8 is a schematic representation depicting an orientation of recording unit with respect to a subject, in accordance with an example implementation of the present subject matter.

FIGS. 9a-9c are schematic representations depicting tracking of the subject by the recording unit, in accordance with an example implementation of the present subject matter.

FIG. 10 is a schematic representation depicting the computation for calculating a distance between the subject and the recording unit, in accordance with an example implementation of the present subject matter.

FIG. 11 illustrates an obstruction detection mechanism, in accordance with an example implementation of the present subject matter.

FIG. 12 illustrates a multipoint zoom feature of a video camera used for recording of seizures, in accordance with an example implementation of the present subject matter.

### DETAILED DESCRIPTION

The present subject matter described herein relates to a system and method for recording of seizures using a video Electroencephalogram (EEG) test.

Video EEG tests are conducted for recording and monitoring seizures in subjects. Conventional video EEG test involves continuous monitoring of the subject and can be done at home or in a hospital setting. An attendant, such as a technologist, caregiver, or the subject, has to activate an event button when a seizure occurs to mark the start of the seizure and has to similarly mark the end of the seizure event. There is also a possibility of the subject going out of focus of a camera of a device that performs video EEG test during seizure attack due to natural movement as well as involuntary movement or falls during the seizures. The camera can generally be refocused by a technologist or a caregiver and hence, typically, a technologist or caregiver has to be present during a video EEG test.

Moreover, conventional video EEG tests use video zooming feature where the subject can be zoomed in or zoomed out in a video for closer look and analysis. However, the video frame always zooms in from the center of the video frame. If the object/area of interest is not at the center, or near the center, zooming in might render the area of interest outside of the video frame. To be able to use the zoom feature later, the attendant has to ensure that the subject is at/near the center of the video frame at all times.

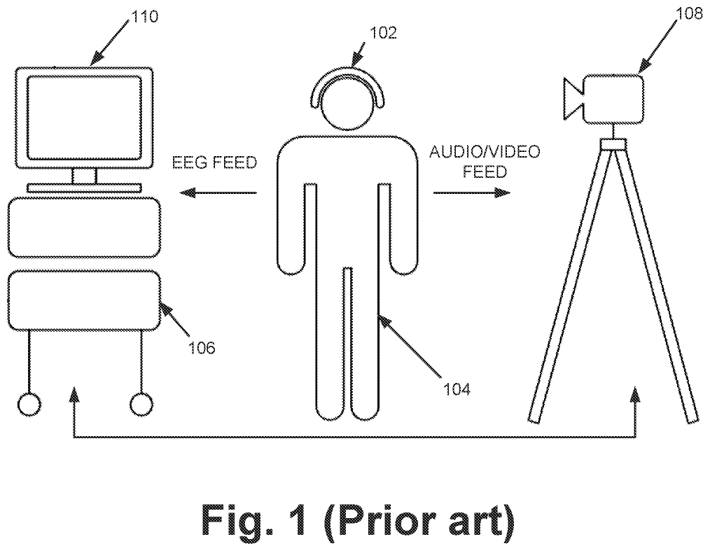

FIG. 1 illustrates a conventional system for recording of seizure using a video EEG test. According to the conventional system, an EEG headset 102 is placed on the scalp of a subject 104. An EEG test set up is established that includes an EEG machine 106 and a video camera 108. The video camera 108 is configured to continuously record the subject 104. The EEG machine 106 can be connected to a computer 110 for receiving and recording EEG feed from EEG headset 102 and video recording from the video camera 108. A caretaker or a technologist may be required for operating an event button (not shown in the figure) for tagging seizure start event and seizure end event, if the subject experiences a seizure. The video camera 108 is typically placed as a standalone device for recording the video and the video feed has to be monitored by the technologist or caregiver to ensure that the subject is in the video frame always.

The present subject matter provides systems and methods for recording of seizures using a video Electroencephalogram (EEG) that overcomes the aforementioned and other problems. The systems and methods of the present subject matter can be used for remotely recording seizure events. In one example, the system includes a recording unit and an EEG headset. The recording unit includes a computing device and an Infrared (IR) sensor that is either integrated with the computing device or connected to the computing device. The IR sensor may continuously monitor coordinates of subject in a frame of reference of the IR sensor, based on signals received from IR transmitters of the EEG headset. Thus, the IR sensor can help in tracking the position of the subject. The computing device may be mounted on a stand which may include a first IMU sensor.

The EEG headset, also referred to as a headset, can include a cap and a tracker. On the tracker, various hardware components, such as a radio transceiver and IR transmitters, for example, IR light emitting diodes (LED), may be mounted. The headset may also include components such as a second IMU sensor and EEG electrodes mounted on the cap. The second IMU sensor, along with the first IMU sensor, helps to track the position and orientation of the subject. The EEG headset can communicate with the computing device using a radio transceiver, for example, using radio signals such as Bluetooth or Wi-Fi or Internet or other wireless communication signals.

In operation, the computing device can receive EEG signals (EEG feed) from the EEG headset, a second IMU signal from the second IMU sensor, recordings from the video camera, and coordinates of the subject wearing the EEG headset detected by the IR sensor. For discussion purposes, the recording received from the video camera is referred to as video recording and coordinates of subject received from the IR sensor are referred to as IR coordinates.

The EEG feed is a waveform received from the EEG headset that reflects an electrical activity of the brain of the subject. Initially, from an EEG feed, a baseline may be established for a subject corresponding to a normal (seizure free) EEG reading for the subject. As the EEG activity may be different for different subjects, a baseline may be established for each subject before continuous monitoring is started for the subject. The baseline may be used as a reference to identify elevated signals, such as a spike in the EEG feed, reflecting the occurrence of a seizure in the subject. The computing device identifies spikes in the EEG feed and/or a jittery motion in the video recording and/or IR coordinates during monitoring of the subject. The computing device then automatically tags the detected spikes and/or the jittery motion as seizure events, without the intervention of the technologist or the caretaker and records it.

In an example, the computing device may be trained over time to accurately identify the seizure events. In some cases, the seizure may not result in any jittery movement but may show spikes in the EEG feed, while in some seizures there may not be any spikes in the EEG feed but there may be jittery movements, and in yet other cases both spikes and jittery movements may be present. From the recordings, a technologist may analyze the jittery movements and EEG feed, and examine the subject to determine the seizure activity. Further, the technologist may provide feedback to the computing device regarding the occurrence of seizures to allow the computing device to learn and improve the accuracy of seizure detection. Therefore, over a period of time, the computing device may be able to accurately tag the seizure events in different subjects with different indicators of the seizures.

The computing device also tracks the orientation of the subject's head using a first IMU signal and a second IMU signal received from the first IMU sensor on the stand and the second IMU sensor on the EEG headset, respectively and may initiate an action to ensure that the video camera always faces the subject for proper video recording. In one example, the computing device may send a notification to a monitoring team to re-orient the recording unit suitably. In another example, the computing device may cause the recording unit to automatically re-orient itself. In various examples, the computing device may also alert the subject or an attendant for example, by sounding a buzzer or alarm. Further, the computing device may also automatically warn the subject or attendant or monitoring team, for example, by sending a notification and/or by sounding a buzzer or alarm, if the subject is too far or too near the IR sensor.

The computing device also tracks the position of the subject based on the subject's movement using the IR coordinates. In an example, the computing device may cause the recording unit to automatically re-orient itself based on the subject's movement such as if the subject were to stand, sit, or fall. In an example, the computing device may send a notification to the monitoring team for reorienting the recording unit. In one example, the computing device can also detect an obstruction between the subject and recording unit based on non-receipt of IR coordinates by the IR sensor and can initiate actions as discussed above.

The present subject matter thus helps in automating video EEG tests by using an automated system having a portable recording unit for performing video EEG test. The portable recording unit helps make the process of video recording and tagging seizure events automatic and may complete the recording remotely without any intervention from a technologist or caregiver.

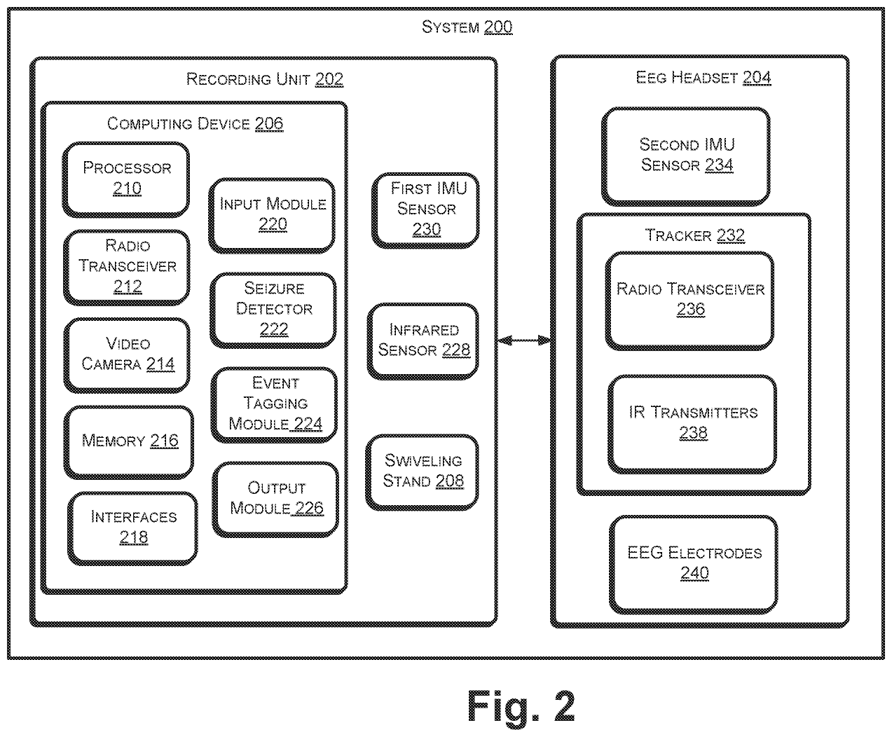

Aspects of the present subject matter are further described in conjunction with FIGS. 2-12. It should be noted that the description and figures merely illustrate the principles of the present subject matter. It will thus be appreciated that various arrangements that embody the principles of the present subject matter, although not explicitly described or shown herein, can be devised from the description and are included within its scope. Moreover, all statements herein reciting principles, aspects, and implementations of the present subject matter, as well as specific examples thereof, are intended to encompass equivalents thereof.

FIG. 2 illustrates a block diagram of a system for recording of seizures, in accordance with an example implementation of the present subject matter. The system 200 includes a recording unit 202 and an EEG headset 204.

In an example, the recording unit 202 can comprise a computing device 206, such as a mobile device, a tablet, a smart display or other smart device, and the like. In one example, the computing device 206 may be mounted on and connected to a stand 208 using mechanisms, such as Universal Serial Bus (USB), firewire, hinge mechanism, and the like. The stand 208 may be a lightweight portable stand usable to hold the computing device to monitor the subject without any intervention from a technologist or a caregiver. In one example, the stand 208 may include a height adjustment mechanism, such as a telescopic stand, or multiple rods that can be detachably coupled to each other to vary the height, and the like. Further, the stand 208 may be a swiveling stand and/or may include a base with wheels for mobility. In yet another example, the stand 208 may be a stationary table top stand.

The computing device 206 can include various hardware components, such as a processor 210, a first radio transceiver 212, a video camera 214, memory 216, interfaces 218 including input/output and network interfaces, and the like. The computing device 206 can also include various modules executable by the processor 210 and that interact with the hardware components. The modules may include an input module 220, a seizure detector module 222, an event tagging module 224, and an output module 226. In one example, the various modules may be stored in the memory 216 along with other software such as operating system and applications used by the computing device 206 for its functioning. In addition, the computing device 206 can include data (not shown in the figure) stored in memory 216. Further, the computing device 206 may also include a display screen (not shown in the figure).

The recording unit 202 further includes an Infrared (IR) sensor 228, either integrated with the computing device 206 or connected to the computing device 206. The IR sensor 228 may be a position sensor and may operate on a coordinate system for continuous monitoring of coordinates of a subject in its frame of reference. The recording unit 202 can also include a first Inertial motion unit (IMU) sensor 230, which may be disposed on the stand 208 to detect the position and orientation of the recording unit 202.

The EEG headset 204, also referred to as headset 204, may be placed on the scalp of a subject for recording and monitoring of seizures. The headset 204 can comprise a cap (not shown in the figure) and a tracker 232 attached to the cap. The tracker 232 may be a wearable accessory, for example, a headband, on which various hardware components may be mounted. The tracker 232 can include a second radio transceiver 236 and IR transmitters 238. The headset 204 can also comprise a second IMU sensor 234 disposed on the cap for generating second IMU signals corresponding to the position of the subject and EEG electrodes 240 disposed on the cap for generating EEG feeds.

In operation, the headset 204 records EEG feeds from the subject's brain using the EEG electrodes 240 and sends the EEG feed to the recording unit 202. Additionally, the IR sensor 228 present in the recording unit 202 can continuously monitor coordinates of subject from the IR transmitters 238 on the headset 204 of the subject. The recording unit 202 can store and analyze the various feeds, including EEG feed, IMU feed, IR coordinates, and video recordings, to detect and automatically record seizure events. For example, the computing device 206 may receive the EEG feed from the EEG headset 204, IR coordinates corresponding to the IR transmitters 238 from the IR sensor 228, and video recording of the subject from the video camera 214. The seizure detector module 222 of the computing device 206 may identify a position of the subject based on at least one of the IR coordinates and the video recording, and cause the computing device to be reoriented based on the position of the subject. The seizure detector module 222 may additionally use the first and second IMU signals for determining the orientation of the subject and cause reorientation of the computing device 206. Further, the seizure detector module 222 may detect seizure events based on the IR coordinates and/or the video recording and/or the EEG feed, and the event tagging module 224 may tag the start and end of the seizure event in the video recordings and EEG feeds. The output module 226 may then provide a seizure event report based on detection of the seizure events. Various example implementations of recording of seizure events by the system 200 are further explained below.

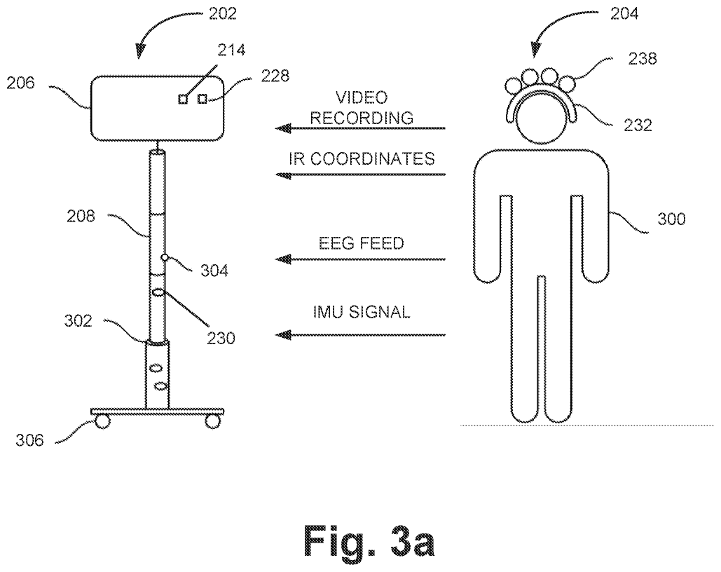

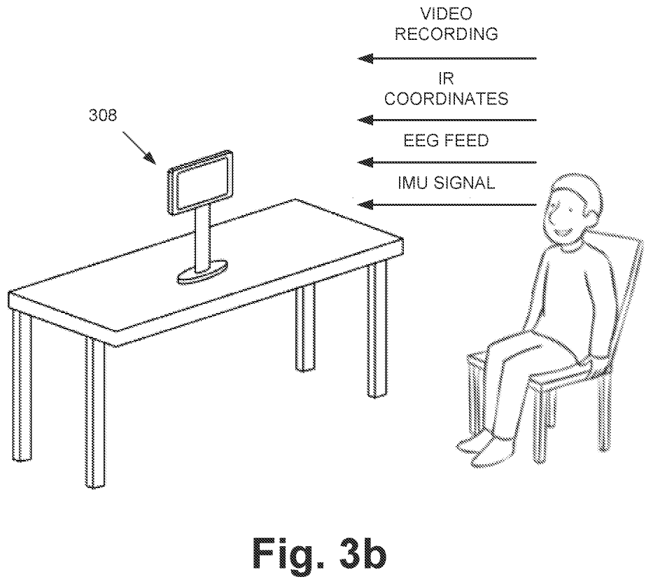

FIG. 3a. illustrates recording of seizure using a recording unit 202, in accordance with an example implementation of the present subject matter. The recording unit 202 includes the computing device 206 mounted on and connected to the stand 208. The computing device 206 can include the video camera 214 for recording the video of a subject 300. In one example, multiple video cameras may be present in the front and/or back of the computing device 206. In one example, the stand 208 may include a height adjustment mechanism 302, such as a telescopic stand, or multiple rods that can be detachably coupled to each other to vary the height, and the like. In an example, the height adjustment can also be done using a hinge mechanism 304. Further, the stand 208 may include a base with wheels 306 for mobility. In an example, the stand 208 may also include motors (not shown in figure) actuatable by the computing device 206 for rotation of wheels along multiple axes to allow it to swivel and change its orientation.

The computing device 206 may be communicably coupled to the EEG headset 204 via the radio transceivers. The EEG headset 204 may include the tracker 232. In an example, the tracker 232 can be worn on head of a subject 300 as shown in the FIG. 3a. In another example, the tracker 232 can be worn on other body parts of the subject 300. The tracker can include the second radio transceiver 236 and the IR transmitters 238, for example, IR light emitting diodes (LED). In an example, the headset 204 includes the second IMU sensor 234 and EEG electrodes 240. It will be understood that the tracker 232 can be made of any suitable shape/form factor and the illustration in the figure is merely for representation purposes. Further, for ease of illustration, the tracker 232 may not be explicitly shown in the remaining figures as being present on the head of the subject, but may be understood to be present.

In operation, for recording the seizures, the input module 220 of the computing device 206 can receive EEG feed from the EEG headset 204 that are recorded using the EEG electrodes 240 of the EEG headset 204. The EEG feed is a waveform output that shows the electrical activity of the brain of the subject 300. When the EEG feed is received, a baseline may be established corresponding to a normal (seizure free) reading for the particular subject. The baseline may be used as a reference to identify elevated signals such as a spike in the EEG feed reflecting the occurrence of a seizure. The seizure detector module 222 can identify spikes in the EEG feed and/or a jittery motion in the video recording or IR coordinates to detect a seizure event. In one example, the seizure detector module 222 is trained using a machine learning model to detect the seizure event. For example, the seizure detector module 222 may be trained using supervised or unsupervised training methods to identify spikes in the EEG waveform that may correspond to seizures and to identify jittery motions in video recordings and from IR coordinates that may correspond to seizures. The training of machine learning models is well known in the art and hence is not explained for brevity. Further, the seizure detector module 222 may receive feedback from a user based on the detection of the seizure event and update the machine learning model based on the feedback.

The event tagging module 224 of the computing device 206 may automatically tag the detected spikes and/or the jittery motion as seizure event and provide it as an output through the output module 226, without the intervention of the technologist or the caretaker. The output module 226 may provide a seizure event report, for example, on a display screen or as a file. In an example, the output module 226 may also cause a notification to be sent to a monitoring team in case a seizure event is detected.

The seizure detector module 222 can also receive first IMU signals from the first IMU sensor 230 of the recording unit 202. In an example, the first IMU sensor 230 may include accelerometer, gyroscope, and compass sensor components. The accelerometer provides an estimate of the velocity of the recording unit, the gyroscope measures rotation of the recording unit along the axis, and the compass measures orientation of the recording unit with respect to the magnetic north-south. The first IMU sensor 230 thus captures the velocity, rotation, and orientation of the recording unit 202.

Further, the seizure detector module 222 can also receive second IMU signals from the second IMU sensor 234 of the headset 204. In an example, the second IMU sensor 234 may also include accelerometer, gyroscope, and compass sensor components that may work in a similar manner to those of the first IMU sensor 230. The second IMU sensor 234 thus captures the velocity, rotation, and orientation of the subject's head. As a result, the second IMU sensor 234 can also provide information indicative of jittery motion of the subject in case of a seizure.

The seizure detector module 222 can receive the first and the second IMU signals from the first IMU sensor 230 and the second IMU sensor 234, respectively to measure the velocity, rotation, and orientation of the subject's head with respect to the recording unit 202. The measurements help to ensure that the subject 300 always faces the video camera 214 for the video recording. In an example, the first and second IMU signals received from the first and the second IMU sensors, respectively may help to check if the relative angle of the headset is equal to the relative angle of the recording unit 202. If the relative angles of headset and the recording unit are the same, the subject 300 may be determined to be facing the video camera 214 for video recording. In an example, if the seizure detector module 222 detects that the orientation of the subject's head is away from the direction required for recording, which may hinder the recording of the video, based on the relative angles, the seizure detector module 222 may send the notification to the monitoring team to reorient the recording unit 202 suitably. In another example, the seizure detector module 222 may cause the recording unit 202 to automatically re-orient itself to face the subject 300. For example, the computing device 206 may cause the stand 208 to swivel for reorientation. In one example, the seizure detector module 222 may also automatically warn the subject 300, for example, by sounding a buzzer or alarm.

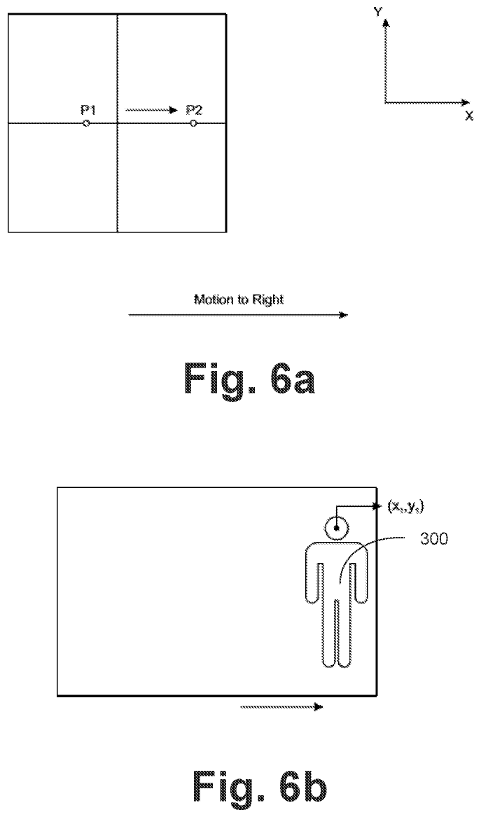

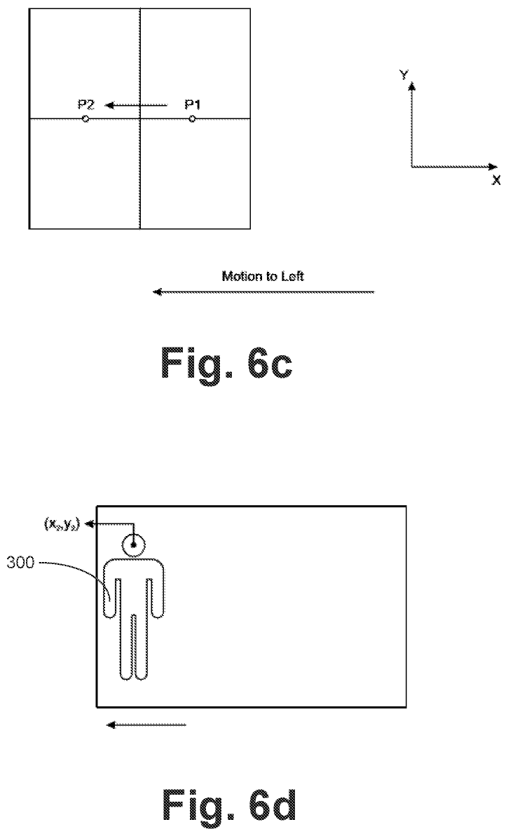

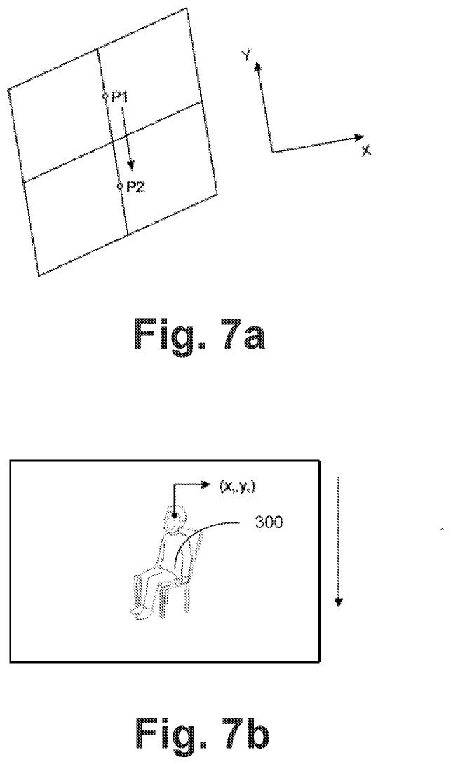

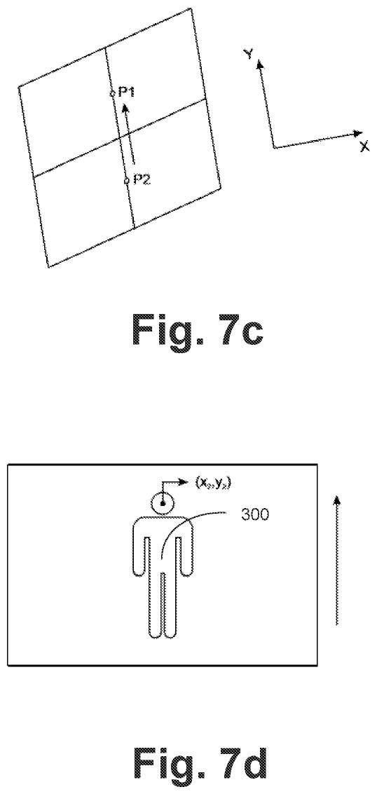

In addition, the seizure detector module 222 may receive IR coordinates of the subject from the IR sensor 228 to detect the position of the subject 300. In an example, the IR sensor 228 includes a coordinate system that can continuously monitor the IR coordinates of the subject in its frame of reference using signals received from the IR transmitters 238 of the headset of the subject. In an example, the IR transmitters of the headset 204 may be visible as dots (referred to as image markers) in the frame of reference of the IR sensor 228. The dots are spread out in X-Y coordinates of the IR coordinate reference frame of the IR sensor 228. The IR sensor 228 may determine the IR coordinates of the image markers, which may be then used by seizure detector module 222 for detecting the change in position of the subject such as horizontal change or vertical change. The detection of change in position of subject will be explained in detail later with reference to FIGS. 6a-6d and 7a-7d. In one example, the seizure detector module 222 can also detect any obstruction between the subject and the IR sensor in case of non-receipt of signals by the IR sensor and initiate actions, such as reorientation of the recording unit 202 or notifying an attendant or the like, as discussed above.

In another example, the recording unit 202 may be stationary and mounted on a table as shown in FIG. 3b. FIG. 3b illustrates a tabletop recording unit 202. The recording unit 202 in this example may be of non-swiveling type. In an example, the tabletop recording unit 202 helps in providing additional portability of the EEG test setup and ease of assembly on a table for performing recording of seizure.

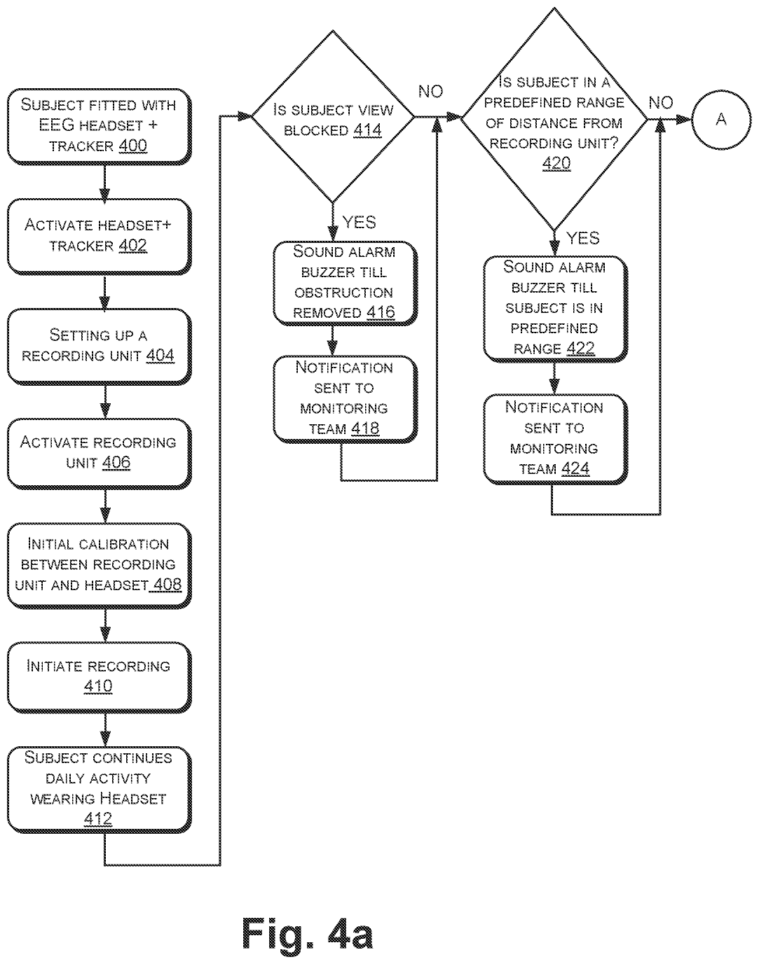

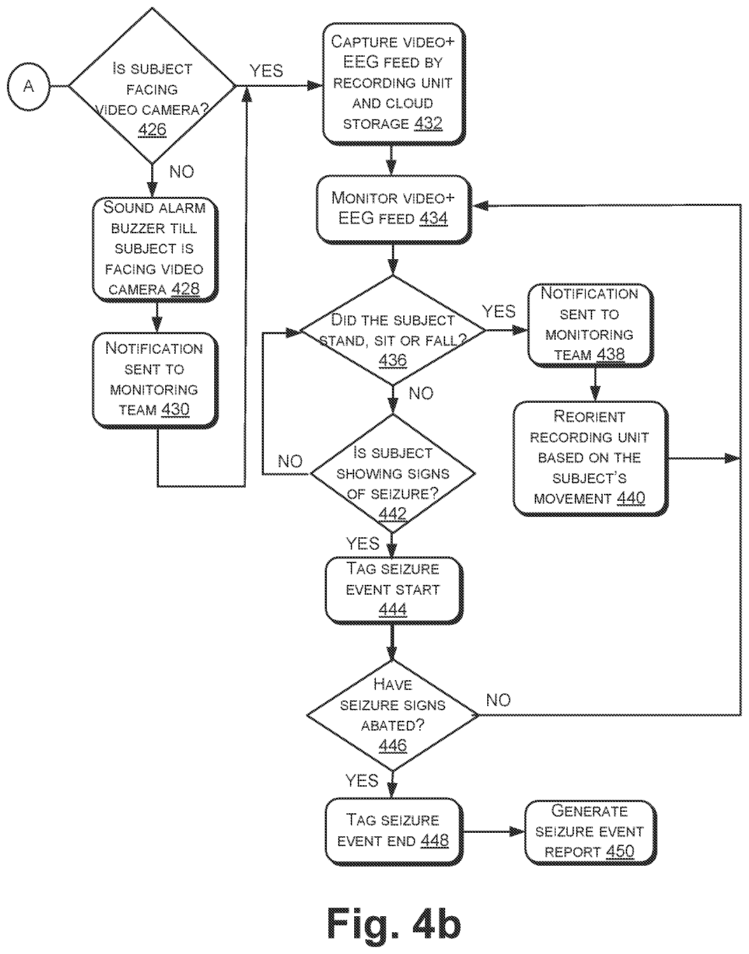

FIGS. 4a and 4b depict a flowchart for recording of seizures, in accordance with an example implementation of the present subject matter. At step 400, the subject is fitted with the EEG headset 204 having a tracker 232. In an example, the tracker 232 can comprise the second radio transceiver 236 and IR transmitters 238, for example IR light emitting diodes (LEDs), and the headset also includes EEG electrodes 240 and second IMU sensor 234.

At step 402, the headset 204 is activated and is kept ready for operation. At step 404, the recording unit 202 is setup in an obstruction free location. At step 406, the recording unit 202 is activated. An initial calibration is performed between the recording unit 202 and the tracker 232 at step 408. At step 410, recording is initiated in the recording unit 202. In an example, the IR coordinates, the video recording, the first IMU signal from the first IMU sensor 230 of the recording unit 202 may be recorded. Further, second IMU signal from the second IMU sensor 234 of the tracker 232 of headset 204 and EEG feed from the headset 204 may also be recorded.

In an example, the video recording is recorded simultaneously along with the EEG feed recording from the EEG headset 204 of the subject. When the EEG feed is received, a baseline may be established corresponding to a normal (seizure free) reading. The baseline may be used as a reference to identify elevated signals, such as spike in the EEG feed, reflecting the occurrence of the seizure.

At step 412, the subject may continue doing their daily activity wearing the headset 204. At step 414, a determination is made to check if the subject's view from the video camera 214 is blocked. This may be determined, for example, by checking if the subjects face is present in the video recording using IMU signals, IR image markers, face recognition in video recording, or a combination thereof. If there is any obstruction, an alarm is sounded at step 416 so that the obstruction is removed. At step 418, a notification can also be sent to a monitoring team for alerting a technologist or an attendant about the obstruction. Further, at step 420, a determination is made to check if the subject 300 is too far or too near the recording unit 202. If the subject 300 is determined to be too far or too near the recording unit 202, an alarm is sounded at step 422 until the subject is in predefined range of distance from the recording unit 202. Further, at step 424, a notification can also be sent to the monitoring team for alerting a technologist or an attendant of the subject's distance being too far or too near the recording unit 202.

Referring to FIG. 4b, at step 426, a determination is made to check if the subject is facing the video camera 214. In an example, the second IMU signal from headset 204 may be used to track the orientation of subject's head and compare it with the first IMU signal received from the recording unit 202 to identify if the subject is facing video camera 214. If the person is not facing the video camera 214, an alarm is sounded at step 428 until the subject is facing the video camera 214. In an example, at step 430, a notification may be sent to the monitoring team for alerting a technologist or an attendant that the subject is not facing the camera. At step 432, when the subject is determined to be facing the video camera 214, the video recording and the EEG feed may be captured by the recording unit 202. For example, the computing device 206 may receive the video recording from the video camera and the EEG feed from the EG headset and may store the video recording and the EEG feed. In an example, the video recording and the EEG feed can also be uploaded to a cloud storage for remote monitoring.

When the video recording and EEG feed are captured at the recording unit 202, the video recording and the EEG feed may be monitored at step 434. In an example implementation, at step 436, a determination is made to check if the subject 300 is standing, siting or has fallen on the ground. If there is any change in the position of subject 300, then a notification is sent to the monitoring team at step 438. The recording unit may be reoriented based on the subject's movement at step 440. In one example, the recording unit may be automatically reoriented on a swiveling telescopic stand. In another example, the recording unit may be manually reoriented.

Further, at step 442, determination is made to check if the subject 300 is showing signs of seizures. In an example, the identification of seizure events is done by the seizure detector module 222. In an example, the seizure detector module 222 may identify spikes in the EEG feed and a jittery motion in the video recording or in the IR coordinates, reflecting the occurrence of a seizure. At step 444, the event tagging module 224 may automatically tag the detected spikes and the jittery motion as seizure events in the EEG feed and video recordings. At step 446, determination is made to check if seizure signs are abated. At step 448, if the seizures are abated, seizure end event is tagged in the video recording and EEG feed. In an example, once seizure events end, the video data and EEG feed for the tagged period is used to generate a seizure event report at step 450. In an example, the seizure event report may be uploaded to the cloud or may be shared with the monitoring team to be made available to a neurologist/doctor or caretaker and the like. In another example, the seizure event report may be displayed on the output module 226 of the recording unit 202. In an example, a stop button can be automatically activated once the seizure event ends to stop the storage of video recording and EEG feed. In one example, while the recording unit 202 may continuously capture/receive and monitor the video recording, EEG feed, IR coordinates, etc., it may store and tag them when a seizure event is detected. In another example, the recording unit 202 may store all the information captured/received for subsequent analysis and feedback for machine learning.

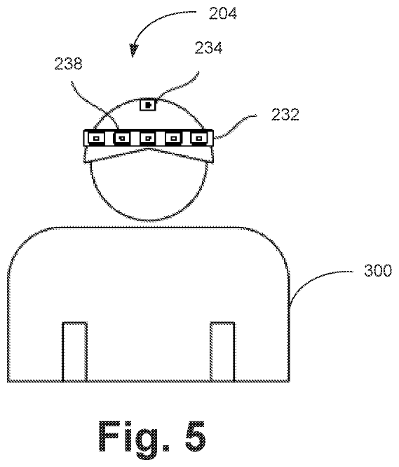

FIG. 5 is a schematic representation of a tracker 232 of an EEG headset 204, in accordance with an example implementation of the present subject matter. The headset 204 can include the tracker 232. The tracker 232 may be a ring like structure on which multiple components, such as a second radio transceiver (not shown in figure), infrared (IR) transmitters 238, for example, IR light emitting diodes (LED), and EEG electrodes 240 may be mounted. In addition, the headset may include a second IMU sensor 234. In an example, the second IMU sensor 234 may be mounted on a cap or on the tracker of the headset. In one example, the tracker may be integrated with a cap of the EEG headset 204. In another example, the tracker can be worn on top of the cap of the EEG headset 204. In various other examples, the tracker 232 can be worn on the subject's arm as an armband or the tracker 232 can be worn as a pendant separate from the cap.

In operation, the EEG electrodes 240 may generate an EEG feed which is a waveform output that shows the electrical activity of the brain of the subject 300. In an example, the EEG feed may be used by the recording unit 202 to detect spikes, reflecting the occurrence of seizure. The second IMU sensor 234 may send a second IMU signal to the recording unit 202 to track the orientation of subject's head. In an example, the recording unit may compare the orientation of subject's head with the orientation of the recording unit based on relative angles using the first and the second IMU signals to check if the subject is facing the video camera 214 of the recording unit for video recording. The second radio transceiver may be used for communicating the first and the second IMU signals, EEG feed, and the like to the recording unit.

FIGS. 6a-6d depict pictorial views of tracking horizontal motion of a subject, in accordance with an example implementation of the present subject matter. FIGS. 6a and 6b show tracking of subject 300 when the subject 300 moves from left to right. In an example, the IR coordinates of the subject may be received from the IR sensor. The IR coordinates may be obtained from multiple IR transmitters, for example IR LEDs. In an example, the IR LEDs of the headset 204 on subject's head are visible as dots (image markers) on the IR coordinate system (frame of reference) of the IR sensor. The dots (image markers) are spread out in X-Y coordinates of the IR coordinate system of the IR sensor 228. In an example, though there are a plurality of IR LEDs on the tracker 232 of headset 204, only one is shown in the figure for ease of understanding. Thus, the tracker 232 shown is represented as a single Infrared LED image marker, which may initially be at position P1 and move horizontally to position P2.

In FIG. 6a, the subject 300 is shown at the initial position P1. When the subject starts moving to right, a new position may be detected as P2. In an example, the new position P2 is along the positive side of X axis of the IR coordinate system of the IR sensor. This implies that the subject has ‘moved right’. Additionally, the tracking by IR coordinates may be followed by augmenting or super-positioning image markers of IR coordinates of subject 300 on image of face or body of the subject received from the video recording of the video camera to avoid inadvertent error as shown in FIG. 6b. In an example, initial position (IR coordinates) of subject on the IR coordinate system may be identified as (x,y) (not shown in figure) and depending on the new coordinate points, (x₁, y₁) as shown in FIG. 6b, it can be concluded that the subject 300 has moved to right.

Similarly, in FIGS. 6c and 6d, tracking of subject 300 when the subject 300 moves from right to left is shown. As mentioned earlier, though there are a plurality of IR LEDs, by way of illustration, only one is shown on IR coordinates for ease of understanding. Thus, the tracker 232 shown is represented as a single Infrared LED image marker at position P1 that moves to position P2. In case of multiple IR image markers, the movement of each of the markers may be identified.

In FIG. 6c, the subject 300 at position P1 may be wearing the tracker 232 and is shown as a single IR light source on the IR coordinate system. When the subject starts moving to left, a new position may be identified as P2. The new position P2 is along the negative side of X axis of the IR coordinate system of the IR sensor 228. This implies that the subject has ‘moved left’. Additionally, the tracking is followed by augmenting image markers of IR coordinates of subject 300 on image of face or body of subject received from the video recording of the video camera to avoid inadvertent error as shown in FIG. 6d. In an example, initial position of the subject on the IR coordinates may be identified as (x, y) (not shown in figure) and depending on the new coordinate points, (x2, y2) as shown in FIG. 6d, it can be concluded that the subject 300 has moved to left. In an example, the seizure detector module 222 may detect the subject's movement using the IR coordinates and may cause the recording unit 202 to automatically re-orient itself based on the subject's movement. In an example, the seizure detector module 222 may also send a notification to a monitoring team for reorienting the recording unit 202.

FIG. 7a-7d depict pictorial views of tracking of vertical motion of a subject, in accordance with an example implementation of the present subject matter. In FIGS. 7a and 7b, tracking of subject 300, when the subject 300 moves downwards or sits down is shown. As discussed above, the tracker 232 is represented as a single Infrared LED image marker (for ease of understanding) at position P1 that moves to position P2.

In FIG. 7a, the subject 300 at position P1 may be wearing the tracker 232 and is shown as a single IR light source on the IR coordinates. The IR coordinates in the frame of reference of the IR sensor may show the initial position of the subject as P1. In an example, when the subject sits down, a new position may be detected as P2. In an example, the new position P2 is along the negative side of Y axis of the IR coordinate system of the IR sensor. This implies that the subject has ‘moved down’. Additionally, the tracking may be followed by augmenting image markers of IR coordinates of subject 300 on image of face or body of subject received from the video recording of the video camera to avoid inadvertent error as shown in FIG. 7b. In an example, initial position of the subject may be identified as (x, y) (not shown in the figure). Further, depending on the new coordinate points, (x₁, y₁) as shown in FIG. 7b, it can be concluded that the subject 300 has moved down or sat down.

Similarly, in FIGS. 7c and 7d, tracking of subject 300 when the subject 300 is moving upwards or standing up is shown. As mentioned earlier, though there are a plurality of IR LEDs, by way of illustration, only one is shown on IR coordinates for better understanding.

In FIG. 7c, the subject 300 at position P2 may be wearing the tracker 232 and is shown as a single IR light source on the IR coordinate system. When the subject starts moving upwards or standing up, a new position is identified as P1. The new position P1 is along the positive side of y axis of the IR coordinate system of the IR sensor 228. This implies that the subject has ‘moved up’ or stood up. Additionally, the tracking is followed by augmenting image markers of IR coordinates of subject 300 on image of face or body of subject received from the video recording of the video camera to avoid any inadvertent error as shown in FIG. 7d. In an example, an initial position of the subject on IR coordinates may be identified as (x, y) (not shown in the figure). Further, depending on the new coordinate points, (x2, y2) as shown in FIG. 7d, it can be concluded that the subject 300 has moved up or stood up from a seated position. In an example, the seizure detector module 222 may detect the subject's movement using the IR coordinates and may cause the recording unit 202 to automatically re-orient itself based on the subject's movement such as subject sitting down or standing up. In an example, the seizure detector module 222 may also send a notification to a monitoring team for reorienting the recording unit.

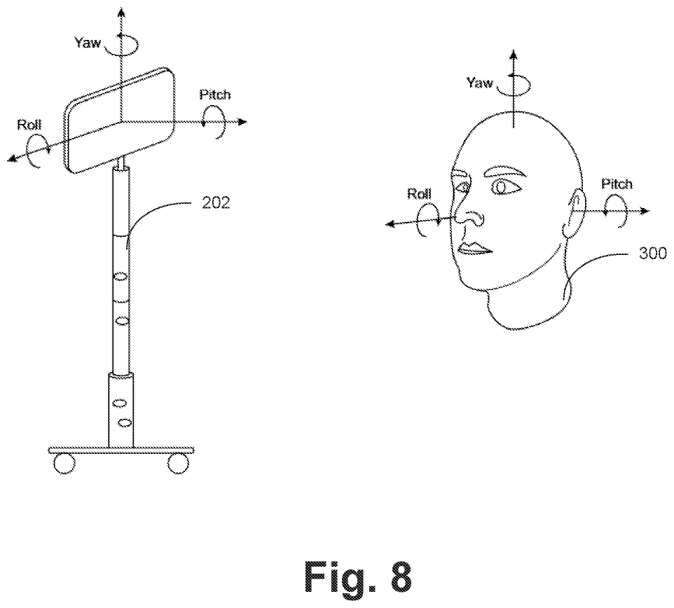

FIG. 8 is a schematic representation depicting an orientation of recording unit 202 with respect to a subject in accordance with an example implementation of the present subject matter. As explained above, the IMU signals are received from the first IMU sensor 230 of the recording unit 202 and the second IMU sensor 234 of the headset 204 worn on the head of the subject. In an example, the first IMU sensor and the second IMU sensor may detect a relative angle between yaw, roll, and pitch of the recording unit 202 and the headset 204. In an example, the yaw, roll, and pitch are detected to measure the rotation and orientation of the subject's head on which the headset is mounted with respect to the recording unit 202.

In an example, the seizure detector module 222 can receive the first and second IMU signals from the first and the second IMU sensors to check if the relative angle of headset (and thereby the head of the subject) is equal to the relative angle of the recording unit 202. If the relative angles of headset and the recording unit are the same, the subject 300 may be determined to be facing the video camera 214 for video recording. In an example, if the seizure detector module 222 detects that the orientation of subject's head is away from the desired position based on the relative angles, the seizure detector module 222 may send a notification to the monitoring team to reorient the recording unit 202 suitably.

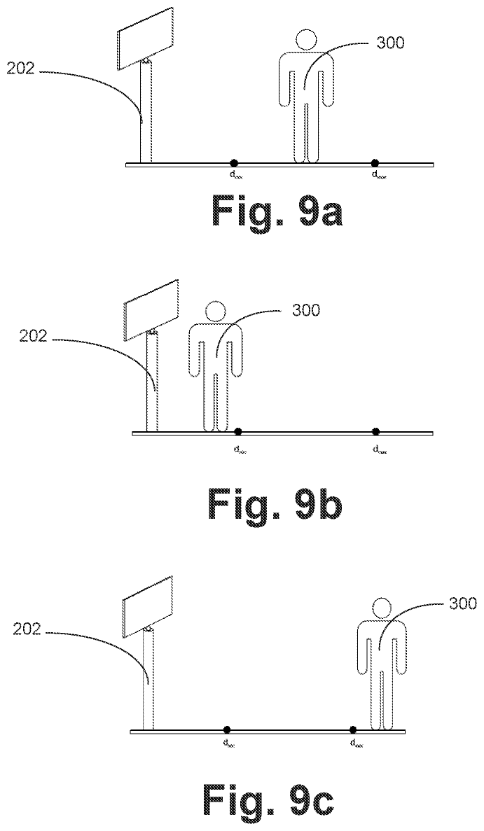

FIG. 9a-9c are schematic representations depicting tracking of the subject by the recording unit 202, in accordance with an example implementation of the present subject matter. FIG. 9a is a schematic representation where a subject 300 is in a predefined range of distance from the recording unit 202. To determine whether the subject 300 is in a desired distance range, an actual distance between the subject 300 and the recording unit 202 is computed, for example, based on the IR signal received by the IR sensor. The computed actual distance is then compared against predetermined minimum and maximum distances (dmin and dmax) corresponding to the predefined distance range. In an example, the predetermined distances may be stored in the memory 216 of the system. In an example, if the computed distance falls in the predefined distance range, the subject 300 is deemed to be at an optimal distance for monitoring.

FIG. 9b is a schematic representation where a subject 300 is too near the recording unit 202. If the subject 300 is too near the recording unit, the video recordings may not be properly captured. Hence, an alert is sent to the monitoring team notifying that the subject 300 is too near the recording unit 202.

FIG. 9c is a schematic representation where a subject is too far from the recording unit 202. If the subject 300 is too far from the recording unit 202, an alert is sent to the monitoring team notifying that the subject 300 is far from the recording unit 202. In an example, the alert is sent to the subject for example, by sounding a buzzer or alarm.

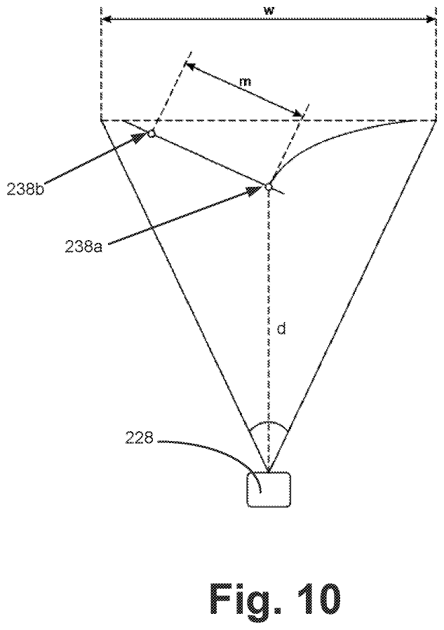

FIG. 10 is a schematic representation depicting the computation for calculating distance between the subject and the recording unit 202 in accordance with an example implementation of the present subject matter. In the FIG. 10, the top view is shown and the tracker 232 for illustration purpose is represented by two IR transceivers corresponding to two IR LED (238a, 238b) at a fixed distance of ‘m’ from each other. The two IR LEDs may correspond to IR transceivers at two ends of the tracker 232, with the IR LED 238a being nearer to the IR sensor 228 than the IR LED 238b.

An angle shown in the figure represents the sensing angle of the IR sensor 228 and ‘w’ represents the horizontal distance that can be sensed by the IR sensor 228. In an example, the value of ‘w’ is variable and is dependent on how far or how near the IR sensor 228 is. Due to the nature of the tracker 232 being worn on the subject 300, it can be at any position relative to the IR sensor 228. For example, the tracker can be worn on different body parts and therefore can be at any position relative to the IR sensor 228. The distance between the headset 204 and the recording unit 202 is computed to be the straight-line distance between the IR sensor 228 of recording unit 202 and the nearest IR LED 238a on the tracker 232. The distance may be represented as ‘d’ in the FIG. 10. After the distance is computed, the computed distance may be compared against predetermined minimum and maximum distances (dmin, dmax). If the computed distance is not in between the predetermined values, an alert is sent to the monitoring team. In an example, the subject 300 is requested to accordingly either come closer to the recording unit 202 or move further away from the recording unit 202.

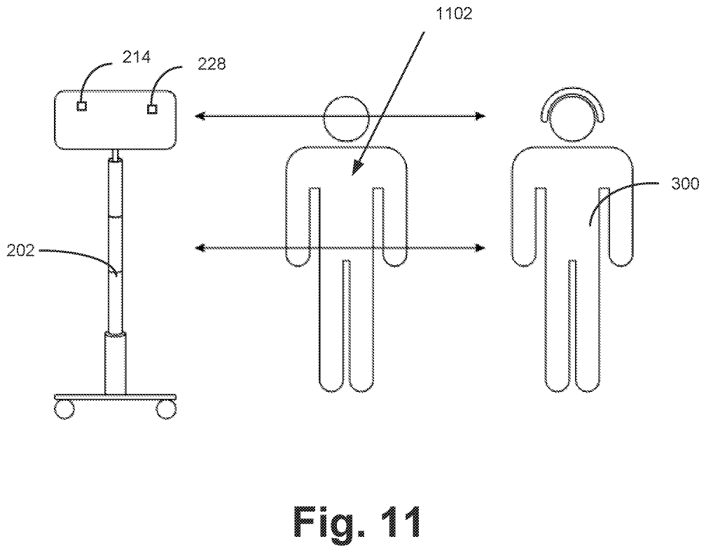

In an example, obstruction detection may also be performed to identify any obstruction/blockage in the path between the subject and the recording unit 202. FIG. 11 illustrates an obstruction detection mechanism in accordance with an example implementation of the present subject matter. In an example scenario, when a person 1102 or an object comes in between the subject 300 and the video camera 214 of the recording unit 202, this may block the view of the subject 300 on the video camera 214 during the video recording. In an example, if the subject 300 experiences seizure during this process, valuable video recording may be lost. Hence, it is significant to warn about the obstruction and to send a notification to clear it. In one example, obstruction can be detected via lack of image markers (or dots) on the IR coordinate system of the IR sensor 228. In the event of obstruction, there will be no image markers on the IR frame of reference of IR sensor 228 and this is construed as obstruction and an alert may be sent to the monitoring team. In an example, an alarm buzz may also be sounded to notify the occurrence of obstruction.

In an example, in addition to above, the recording unit 202 may also be integrated with a multipoint zoom feature which is designed to give multiple and simultaneous (via split screen) views of seizure events to a neurologist/doctor.

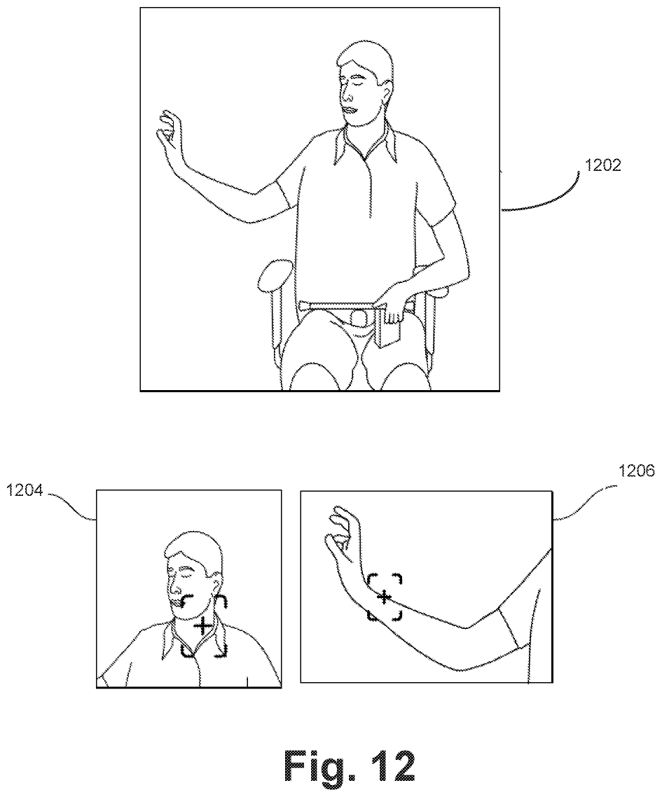

FIG. 12 illustrates a multipoint zoom feature of video camera 214 in accordance with an example implementation of the present subject matter. Multipoint zoom feature offers to zoom a video from any point of a video frame 1202. This feature allows the neurologist/doctor to focus and zoom on any specific area in the video. In an example, if the neurologist wishes to zoom and focus on the head and hand. The video frame splits into to two views with the head zoomed in on a left split video 1204 and hand is zoomed on a right split video 1206.

Thus, using a lightweight, simple and an automated portable recording unit for video Electroencephalogram (EEG) test, recording and monitoring of seizures in a subject can be performed with minimal intervention of technologist or caregiver. The orientation of the subject's head may be tracked based on the relative angles to ensure that the subject always faces the video camera for proper video recording. Further, obstruction between the subject and the camera may be detected and action may be taken to remove the obstruction from the field of view. Additionally, the subject may be warned by sounding a buzzer alarm, if the subject is too far or too near the IR sensor. Further, with the multipoint zoom feature of video camera, any area of interest in the video frame can be focused, thus improving the monitoring of the subject.

Although implementations for methods and systems for recording of seizures have been described in a specific to structural features and/or methods, it is to be understood that the present subject matter is not necessarily limited to the specific features or methods described. Rather, the specific features and methods are disclosed and explained in the context of a few example implementations.

I/We claim:

1. A system for recording of seizures, the system comprising:
an electroencephalogram (EEG) headset to be worn by a subject, the EEG headset comprising EEG electrodes for generating an EEG feed and InfraRed (IR) transmitters; and
a recording unit to be communicably coupled to the EEG headset, wherein the recording unit comprises a computing device for receiving EEG feed from the EEG headset, IR coordinates corresponding to the IR transmitters from an IR sensor, and video recording of the subject from a video camera, wherein the computing device comprises:
a seizure detector module to identify a position of the subject based on the IR coordinates or the video recording or a combination thereof; cause the computing device to be reoriented based on the position of the subject; and detect a seizure event based on the EEG feed or video recording or IR coordinates or a combination thereof; and
an output module to provide a seizure event report based on detection of the seizure event.

2. The system as claimed in claim 1, wherein the recording unit comprises a stand and wherein the computing device is mounted on the stand.

3. The system as claimed in claim 2, wherein the stand is telescopic and swiveling for automatic reorientation of the stand based on the position of the subject.

4. The system as claimed in claim 2, comprising a first IMU sensor disposed on the stand and a second IMU sensor disposed on the EEG headset, wherein the seizure detector module is to receive a first IMU signal from the first IMU sensor and a second IMU signal from the second IMU sensor for detecting the orientation of the subject with respect to the recording unit and to provide a notification based on the orientation of the subject.

5. The system as claimed in claim 1, wherein the seizure detector module is to identify at least one of spikes in the EEG feed, a jittery motion in the video recording, and jittery motion in the IR coordinates to detect the seizure event.

6. The system as claimed in claim 5, comprising an event tagging module to automatically tag the instances of detection of the spikes and the jittery motions as seizure events in the video recording.

7. The system as claimed in claim 1, wherein the seizure detector module is to augment image markers of IR coordinates on a face or a body of the subject received from the video recording to track the position of the subject.

8. The system as claimed in claim 1, wherein the seizure detector module is to detect an obstruction in a path between the subject and the recording unit based on non-receipt of the IR coordinates from the IR sensor.

9. The system as claimed in claim 1, wherein the seizure detector module is to determine a distance of the subject from the recording unit based on the IR coordinates.

10. The system as claimed in claim 1, wherein the seizure detector module is to determine a horizontal and a vertical motion of the subject from the recording unit based on the IR coordinates and video recording.

11. The system as claimed in claim 1, wherein the seizure detector module is trained using a machine learning model to detect the seizure event and wherein the seizure detector module is to receive feedback from a user based on the detection of the seizure event and update the machine learning model based on the feedback.

12. The system as claimed in claim 1, wherein the recording unit and the EEG headset comprise radio transceivers for communication.
